SEQUENCE LISTING (158 sequences)

SEQ ID NO: 1 - DNA, 49 bp, Artificial Sequence
  Chemically synthesized primer sequence for subcloning RGT2-up
catcgacaagcttatgggtttagaagacagtgctctcttgcaaaagtac

SEQ ID NO: 2 - DNA, 49 bp, Artificial Sequence
  Chemically synthesized primer sequence for subcloning RGT2-down
gtcgatgggatccctatacagaggcttcttcaacttcagaaacaccttc

SEQ ID NO: 3 - DNA, 52 bp, Artificial Sequence
  Chemically synthesized primer sequence for subcloning SNF3-up
catcgacgaattcatgtggaaatttctagaagctctactttacgacaacacc

SEQ ID NO: 4 - DNA, 51 bp, Artificial Sequence
  Chemically synthesized primer sequence for subcloning SNF3-down
gtcgatgagatctctattcttcttcgtcctcgtcttcgtcttcttcatcgg

SEQ ID NO: 5 - DNA, 44 bp, Artificial Sequence
  Chemically synthesized primer sequence for subcloning SUT1-up
catcgacgaattcatgtcttctcaagatattccttcaggtgttc

SEQ ID NO: 6 - DNA, 43 bp, Artificial Sequence
  Chemically synthesized primer sequence for subcloning SUT1-down
gtcgatgaagcttttaaacatgttcgtcaacaggcttttcatc

SEQ ID NO: 7 - DNA, 49 bp, Artificial Sequence
  Chemically synthesized primer sequence for subcloning SUT234-up
catcgacgaattcatgtcctcacaagatttaccctcgggtgctcaaacc

SEQ ID NO: 8 - DNA, 48 bp, Artificial Sequence
  Chemically synthesized primer sequence for subcloning SUT23-down
cgatgggatcctaaacttgctcttgctcttttgcttcggcaccataag

SEQ ID NO: 9 - DNA, 51 bp, Artificial Sequence
  Chemically synthesized primer sequence for subcloning SUT4-down
gtcgatgggatccctaaacttgctcttgctcttttgtttcggcaccataag

SEQ ID NO: 10 - DNA, 35 bp, Artificial Sequence
  Chemically synthesized primer sequence for subcloning SUT2-up
ttgagaatttcattgaagcatatcgaagtatctcc

SEQ ID NO: 11 - DNA, 35 bp, Artificial Sequence
  Chemically synthesized primer sequence for subcloning SUT3-up
ttaggatacttcaccttatgtacataagtgcctcc

SEQ ID NO: 12 - DNA, 35 bp, Artificial Sequence
  Chemically synthesized primer sequence for subcloning SUT4-up
ttgagatattcattgaagcatatctaaatacctcc

SEQ ID NO: 13 - DNA, 45 bp, Artificial Sequence
  Chemically synthesized primer sequence for subcloning XUT1-up
catcgacgaattcatgcacggtggtggtgacggtaacgatatcac

SEQ ID NO: 14 - DNA, 44 bp, Artificial Sequence
  Chemically synthesized primer sequence for subcloning XUT1-down
gtcgatgggatccttatttttcaacgtggtagacatcagccttg

SEQ ID NO: 15 - DNA, 36 bp, Artificial Sequence
  Chemically synthesized primer sequence for subcloning XUT1-delta-EcoRI-S
ctcttgttcaaatggaatttcttgaaatgaaggcac

SEQ ID NO: 16 - DNA, 36 bp, Artificial Sequence
  Chemically synthesized primer sequence for subcloning XUT1-delta-EcoRI-AS
gtgccttcatttcaagaaattccatttgaacaagag

SEQ ID NO: 17 - DNA, 43 bp, Artificial Sequence
  Chemically synthesized primer sequence for subcloning XUT2-up
catcgacgaattcatgaagtattttcaaatctggaaatcaggc

SEQ ID NO: 18 - DNA, 44 bp, Artificial Sequence
  Chemically synthesized primer sequence for subcloning XUT2-down
gtcgatgggatcctatgagaacaaatcgtcaatggcttctaaag

SEQ ID NO: 19 - DNA, 44 bp, Artificial Sequence
  Chemically synthesized primer sequence for subcloning XUT3-up
catcgacaagcttatgagagaagttggtattcttgatgttgccc

SEQ ID NO: 20 - DNA, 44 bp, Artificial Sequence
  Chemically synthesized primer sequence for subcloning XUT3-down
gtcgatgggatccttattctgacatttcaatcgagttgcgatcc

SEQ ID NO: 21 - DNA, 37 bp, Artificial Sequence
  Chemically synthesized primer sequence for subcloning XUT3-delta-HindIII-S
ggtcgcgacgaagaagcatggagctctttgaaatatc

SEQ ID NO: 22 - DNA, 37 bp, Artificial Sequence
  Chemically synthesized primer sequence for subcloning XUT3-delta-HindIII-AS
gatatttcaaagagctccatgcttcttcgtcgcgacc

SEQ ID NO: 23 - DNA, 43 bp, Artificial Sequence
  Chemically synthesized primer sequence for subcloning XUT4-up
catcgacgaattcatgtcttcgttattgactaacgaatacttc

SEQ ID NO: 24 - DNA, 45 bp, Artificial Sequence
  Chemically synthesized primer sequence for subcloning XUT4-down
cgatgaagcttattccatctcattcaacttgtacttaaagatttc

SEQ ID NO: 25 - DNA, 45 bp, Artificial Sequence
  Chemically synthesized primer sequence for subcloning XUT5-up
catcgacgaattcatgacggaaagaagcattggacctttaatccc

SEQ ID NO: 26 - DNA, 45 bp, Artificial Sequence
  Chemically synthesized primer sequence for subcloning XUT5-down
gtcgatgagatctttacttctttgtattaacaacaaaaccttgtc

SEQ ID NO: 27 - DNA, 45 bp, Artificial Sequence
  Chemically synthesized primer sequence for subcloning XUT6-up
catcgacgaattcatgtccagtgttgaaaaaagtgctgaaactgc

SEQ ID NO: 28 - DNA, 45 bp, Artificial Sequence
  Chemically synthesized primer sequence for subcloning XUT6-down
gtcgatgagatctttagctgatgttttcgacatgctctattctag

SEQ ID NO: 29 - DNA, 45 bp, Artificial Sequence
  Chemically synthesized primer sequence for subcloning XUT7-up
tcgacgaattcatgacttttgcagttaacttgtatgtgtttgcag

SEQ ID NO: 30 - DNA, 44 bp, Artificial Sequence
  Chemically synthesized primer sequence for subcloning XUT7-down
gtcgatgaagcttagtccaaatcgtccaaatcgtccatttctac

SEQ ID NO: 31 - DNA, 45 bp, Artificial Sequence
  Chemically synthesized primer sequence for subcloning HXT2.1-up
cgacgaattcatgttgcatatttttgtgtttttatgtactctttc

SEQ ID NO: 32 - DNA, 43 bp, Artificial Sequence
  Chemically synthesized primer sequence for subcloning HXT2.1-down
cgatgggatcctcagacgactcttccgatctctgcggaagaag

SEQ ID NO: 33 - DNA, 41 bp, Artificial Sequence
  Chemically synthesized primer sequence for subcloning HXT2.2-up
tcgaccaattgatgtctaagaatcaaacaatcaaagaccag

SEQ ID NO: 34 - DNA, 40 bp, Artificial Sequence
  Chemically synthesized primer sequence for subcloning HXT2.2-down
cgatgggatcctcaaacactttttccgacttcaacagaag

SEQ ID NO: 35 - DNA, 45 bp, Artificial Sequence
  Chemically synthesized primer sequence for subcloning HXT2.3-up
gacgaattcatgtggtggaagcataagcacttcagatttttaaac

SEQ ID NO: 36 - DNA, 45 bp, Artificial Sequence
  Chemically synthesized primer sequence for subcloning HXT2.3-down
gatgggatcctagacagtcttggaagctatatccacgctttcttc

SEQ ID NO: 37 - DNA, 44 bp, Artificial Sequence
  Chemically synthesized primer sequence for subcloning HXT2.4-up
tcgacaagcttatgtctgacaaacttcacaacatcaaggatcag

SEQ ID NO: 38 - DNA, 45 bp, Artificial Sequence
  Chemically synthesized primer sequence for subcloning HXT2.4-down
cgatgggatccttaaactctttcaccaatcttctcgtttgaagag

SEQ ID NO: 39 - DNA, 45 bp, Artificial Sequence
  Chemically synthesized primer sequence for subcloning HXT2.6-up
cgacaagcttatgtcccaactgaaagaaaaatcaaacgttattac

SEQ ID NO: 40 - DNA, 45 bp, Artificial Sequence
  Chemically synthesized primer sequence for subcloning HXT2.6-down
gatgggatcctaggcagaacttccacgttcagaatggacaatatc

SEQ ID NO: 41 - DNA, 45 bp, Artificial Sequence
  Chemically synthesized primer sequence for subcloning HXT4-up
tcgacaagcttatgagcattgagaccgattttattgaaattgagc

SEQ ID NO: 42 - DNA, 42 bp, Artificial Sequence
  Chemically synthesized primer sequence for subcloning HXT4-down
cgatgggatcctagctgaaaccccagaaccttttcacgttac

SEQ ID NO: 43 - DNA, 45 bp, Artificial Sequence
  Chemically synthesized primer sequence for subcloning HGT1-up
cgaccaattgatgggcttgaaaatctacaatctgtatggaatcac

SEQ ID NO: 44 - DNA, 45 bp, Artificial Sequence
  Chemically synthesized primer sequence for subcloning HGT1-down
gatgggatcctaattaaaattgaatattgagtataactcatattc

SEQ ID NO: 45 - DNA, 52 bp, Artificial Sequence
  Chemically synthesized primer sequence for subcloning HGT1-delta-Intron-S
gtctagcaatggtattggtgcagttgttggttgtgttatttgtggaatctac

SEQ ID NO: 46 - DNA, 52 bp, Artificial Sequence
  Chemically synthesized primer sequence for subcloning HGT1-delta-Intron-AS
gtagattccacaaataacacaaccaacaactgcaccaataccattgctagac

SEQ ID NO: 47 - DNA, 45 bp, Artificial Sequence
  Chemically synthesized primer sequence for subcloning HGT2-up
atcgacaagcttatgagctacgaagataaactcgttcaacctgcc

SEQ ID NO: 48 - DNA, 45 bp, Artificial Sequence
  Chemically synthesized primer sequence for subcloning HGT2-down
tcgatgggatccttaaggcttttcctcagaacttgactcggcatc

SEQ ID NO: 49 - DNA, 45 bp, Artificial Sequence
  Chemically synthesized primer sequence for subcloning STL1-up
atcgacgaattcatggcatatcttgattggttaacagctagaacc

SEQ ID NO: 50 - DNA, 44 bp, Artificial Sequence
  Chemically synthesized primer sequence for subcloning STL1-down
gtcgatgaagcttaagagctgctggatgcgttttctttggtttc

SEQ ID NO: 51 - DNA, 45 bp, Artificial Sequence
  Chemically synthesized primer sequence for subcloning MFS5-up
atcgacgaattcatgattggaacaaaagatattaaagaaaataac

SEQ ID NO: 52 - DNA, 45 bp, Artificial Sequence
  Chemically synthesized primer sequence for subcloning MSF5-down
tcgatgggatccttatgtttccgatgtcttagatttagtatcttc

SEQ ID NO: 53 - DNA, 44 bp, Artificial Sequence
  Chemically synthesized primer sequence for subcloning AUT1-up
tcgacgaattcatgagtgctgacgaaaaagtcgctgctgccggc

SEQ ID NO: 54 - DNA, 45 bp, Artificial Sequence
  Chemically synthesized primer sequence for subcloning AUT1-down
cgatgggatcctactcgacataagagacttctggcttgtcgttcc

SEQ ID NO: 55 - DNA, 44 bp, Artificial Sequence
  Chemically synthesized primer sequence for subcloning FUC1-up
tcgacgaattcatgttggatacgttgaatgcccacttccaaacc

SEQ ID NO: 56 - DNA, 45 bp, Artificial Sequence
  Chemically synthesized primer sequence for subcloning FUC1-down
gatgagatctaaacgtagacagcataggtgcttgctaatatatag

SEQ ID NO: 57 - DNA, 45 bp, Artificial Sequence
  Chemically synthesized primer sequence for subcloning MAL1-up
tcgacaagcttatggttactgaatacgtcgaagacgtaaagaatc

SEQ ID NO: 58 - DNA, 44 bp, Artificial Sequence
  Chemically synthesized primer sequence for subcloning MAL1-down
cgatgggatcctaagccttttcaaagatttcttcttcttcagcc

SEQ ID NO: 59 - DNA, 45 bp, Artificial Sequence
  Chemically synthesized primer sequence for subcloning MAL2-up
tcgacaagcttatggttagtgaatacgtcgaagacataaagaacc

SEQ ID NO: 60 - DNA, 44 bp, Artificial Sequence
  Chemically synthesized primer sequence for subcloning MAL2-down
cgatgagatctctaagtagagacctggaccttttcaatgatatc

SEQ ID NO: 61 - DNA, 45 bp, Artificial Sequence
  Chemically synthesized primer sequence for subcloning MAL3-up
tcgacaagcttatgagctcggtgtttgttgaagatataaaaaatc

SEQ ID NO: 62 - DNA, 42 bp, Artificial Sequence
  Chemically synthesized primer sequence for subcloning MAL3-down
cgatgagatctcaacggttttgttcaagaatatcataattgg

SEQ ID NO: 63 - DNA, 45 bp, Artificial Sequence
  Chemically synthesized primer sequence for subcloning MAL4-up
tcgacaagcttatgtcgactgagtacgttgaagacaacaagaacc

SEQ ID NO: 64 - DNA, 44 bp, Artificial Sequence
  Chemically synthesized primer sequence for subcloning MAL4-down
cgatgagatcttaagccttttcaaagatttcgttgttgtcatcc

SEQ ID NO: 65 - DNA, 45 bp, Artificial Sequence
  Chemically synthesized primer sequence for subcloning MAL5-up
gacgaattcatgacacttagtgaagcgtggaaagatcacaaaccg

SEQ ID NO: 66 - DNA, 44 bp, Artificial Sequence
  Chemically synthesized primer sequence for subcloning MAL5-down
cgatgggatccttattcttcaaacttttcgatttcagaagtatc

SEQ ID NO: 67 - DNA, 45 bp, Artificial Sequence
  Chemically synthesized primer sequence for subcloning QUP1-up
tcgacaagcttatgagcttcagcaacaaatcaactgcatccacgc

SEQ ID NO: 68 - DNA, 43 bp, Artificial Sequence
  Chemically synthesized primer sequence for subcloning QUP1-down
cgatgagatcttaaaccttgttaggcaccaaaatagagtcgtc

SEQ ID NO: 69 - DNA, 45 bp, Artificial Sequence
  Chemically synthesized primer sequence for subcloning QUP2-up
tcgacgaattcatggttgttgtagcacttacagctgcgtttgcag

SEQ ID NO: 70 - DNA, 45 bp, Artificial Sequence
  Chemically synthesized primer sequence for subcloning QUP2-down
gtcgatgggatcctactcatttgattcttctcgagaacgcccccg

SEQ ID NO: 71 - DNA, 43 bp, Artificial Sequence
  Chemically synthesized primer sequence for subcloning QUP3-up
cgacgaattcatgggccatgatgattctccacctcagatctac

SEQ ID NO: 72 - DNA, 42 bp, Artificial Sequence
  Chemically synthesized primer sequence for subcloning QUP3-down
cgatgggatcctaaagcttcttttcgttttctttgtcttcag

SEQ ID NO: 73 - DNA, 50 bp, Artificial Sequence
  Chemically synthesized primer sequence for subcloning QUP3-delta-Intron-S
gcaagaggttatgacgaagggtgcatatctggtaatatcactctaccatc

SEQ ID NO: 74 - DNA, 50 bp, Artificial Sequence
  Chemically synthesized primer sequence for subcloning QUP3-delta-Intron-AS
gatggtagagtgatattaccagatatgcacccttcgtcataacctcttgc

SEQ ID NO: 75 - DNA, 44 bp, Artificial Sequence
  Chemically synthesized primer sequence for subcloning QUP4-up
cgacgaattcatgagtattgaaaagctcaataacgttgttgcag

SEQ ID NO: 76 - DNA, 45 bp, Artificial Sequence
  Chemically synthesized primer sequence for subcloning QUP4-down
cgatgggatcctatttgtacttgttgttcttgtcttcatcactag

SEQ ID NO: 77 - DNA, 49 bp, Artificial Sequence
  Chemically synthesized primer sequence for subcloning QUP4-delta-Intron-S
gaaatctataacaagactcttttcctttctgtcttggtttttggtattc

SEQ ID NO: 78 - DNA, 49 bp, Artificial Sequence
  Chemically synthesized primer sequence for subcloning QUP4-delta-Intron-AS
gaataccaaaaaccaagacagaaaggaaaagagtcttgttatagatttc

SEQ ID NO: 79 - DNA, 42 bp, Artificial Sequence
  Chemically synthesized primer sequence for subcloning LAC1-up
tcgacaagcttatgtcctctgaaatgcttagcaaaagtgaag

SEQ ID NO: 80 - DNA, 42 bp, Artificial Sequence
  Chemically synthesized primer sequence for subcloning LAC1-down
cgatgggatcctcaattcattccagactttcttctagaaacg

SEQ ID NO: 81 - DNA, 45 bp, Artificial Sequence
  Chemically synthesized primer sequence for subcloning LAC2-up
tcgacaagcttatgagcacaaatagtttaaacgattcgtacaatc

SEQ ID NO: 82 - DNA, 45 bp, Artificial Sequence
  Chemically synthesized primer sequence for subcloning LAC2-down
gatgggatccttaggcaacataattcttaccgagcttaagattag

SEQ ID NO: 83 - DNA, 45 bp, Artificial Sequence
  Chemically synthesized primer sequence for subcloning LAC3-up
gacgaattcatgttgtatacgatatgtgctggtttatatctttgc

SEQ ID NO: 84 - DNA, 44 bp, Artificial Sequence
  Chemically synthesized primer sequence for subcloning LAC3-down
cgatgggatcctcagctatttgtcttttgctctgtatattcttc

SEQ ID NO: 85 - DNA, 51 bp, Artificial Sequence
  Chemically synthesized primer sequence for subcloning LAC3-delta-Intron-S
cataccgtgaaagagagtatcaagtttaataagagaaaagaagagataatg

SEQ ID NO: 86 - DNA, 51 bp, Artificial Sequence
  Chemically synthesized primer sequence for subcloning LAC3-delta-Intron-AS
cattatctcttcttttctcttattaaacttgatactctctttcacggtatg

SEQ ID NO: 87 - DNA, 22 bp, Artificial Sequence
  Chemically synthesized primer sequence for Realtime-PCR RGT2-RTP-up
ttcgtgtggtaccttgtctacg

SEQ ID NO: 88 - DNA, 21 bp, Artificial Sequence
  Chemically synthesized primer sequence for Realtime-PCR RGT2-RTP-down
cggatggaatgaatctggtag

SEQ ID NO: 89 - DNA, 21 bp, Artificial Sequence
  Chemically synthesized primer sequence for Realtime-PCR SNF3-RTP-up
catagcaacatcaccagcaac

SEQ ID NO: 90 - DNA, 21 bp, Artificial Sequence
  Chemically synthesized primer sequence for Realtime-PCR SNF3-RTP-down
tcgtcttcgtcttcttcatcg

SEQ ID NO: 91 - DNA, 21 bp, Artificial Sequence
  Chemically synthesized primer sequence for Realtime-PCR SUT1-RTP-up
gaattgtacgcttccaacgtg

SEQ ID NO: 92 - DNA, 21 bp, Artificial Sequence
  Chemically synthesized primer sequence for Realtime-PCR SUT1-RTP-down
gcttttcatcagcagcgtaac

SEQ ID NO: 93 - DNA, 20 bp, Artificial Sequence
  Chemically synthesized primer sequence for Realtime-PCR SUT2/3/4-RTP-up
aaccaaaggtttgtcgttgg

SEQ ID NO: 94 - DNA, 21 bp, Artificial Sequence
  Chemically synthesized primer sequence for Realtime-PCR SUT2/3/4-RTP-down
gcaccataagtggagttttgc

SEQ ID NO: 95 - DNA, 21 bp, Artificial Sequence
  Chemically synthesized primer sequence for Realtime-PCR HXT2.1-RTP-up
ggtgacgatccaaactcttcc

SEQ ID NO: 96 - DNA, 21 bp, Artificial Sequence
  Chemically synthesized primer sequence for Realtime-PCR HXT2.1-RTP-down
tcagacgactcttccgatctc

SEQ ID NO: 97 - DNA, 22 bp, Artificial Sequence
  Chemically synthesized primer sequence for Realtime-PCR HXT2.2-RTP-up
gacgtttgagttggtcattgtc

SEQ ID NO: 98 - DNA, 21 bp, Artificial Sequence
  Chemically synthesized primer sequence for Realtime-PCR HXT2.2-RTP-down
tgccattgccagtatctaagg

SEQ ID NO: 99 - DNA, 21 bp, Artificial Sequence
  Chemically synthesized primer sequence for Realtime-PCR HXT2.3-RTP-up
caagagctttcggtgatgatg

SEQ ID NO: 100 - DNA, 21 bp, Artificial Sequence
  Chemically synthesized primer sequence for Realtime-PCR HXT2.3-RTP-down
gctatatccacgctttcttcg

SEQ ID NO: 101 - DNA, 21 bp, Artificial Sequence
  Chemically synthesized primer sequence for Realtime-PCR HXT2.4-RTP-up
aaaccttcggttacaccttgg

SEQ ID NO: 102 - DNA, 21 bp, Artificial Sequence
  Chemically synthesized primer sequence for Realtime-PCR HXT2.4-RTP-down
gtcttccaagtgttcgacagc

SEQ ID NO: 103 - DNA, 21 bp, Artificial Sequence
  Chemically synthesized primer sequence for Realtime-PCR HXT2.6-RTP-up
tcggatacacgttggaagaag

SEQ ID NO: 104 - DNA, 21 bp, Artificial Sequence
  Chemically synthesized primer sequence for Realtime-PCR HXT2.6-RTP-down
gaacttccacgttcagaatgg

SEQ ID NO: 105 - DNA, 20 bp, Artificial Sequence
  Chemically synthesized primer sequence for Realtime-PCR HXT4-RTP-up
ggtggtatcgctttcattgg

SEQ ID NO: 106 - DNA, 22 bp, Artificial Sequence
  Chemically synthesized primer sequence for Realtime-PCR HXT4-RTP-down
gcaacttggaaagaagacttgg

SEQ ID NO: 107 - DNA, 21 bp, Artificial Sequence
  Chemically synthesized primer sequence for Realtime-PCR XUT1-RTP-up
tgaacttttcggtgacacctc

SEQ ID NO: 108 - DNA, 21 bp, Artificial Sequence
  Chemically synthesized primer sequence for Realtime-PCR XUT1-RTP-down
acgtggtagacatcagccttg

SEQ ID NO: 109 - DNA, 21 bp, Artificial Sequence
  Chemically synthesized primer sequence for Realtime-PCR XUT2-RTP-up
cttcagcagttggagcttttg

SEQ ID NO: 110 - DNA, 21 bp, Artificial Sequence
  Chemically synthesized primer sequence for Realtime-PCR XUT2-RTP-down
gccgaagacaagttggaaaac

SEQ ID NO: 111 - DNA, 21 bp, Artificial Sequence
  Chemically synthesized primer sequence for Realtime-PCR XUT2-RTP-up
cttatcatcccggaaaccaag

SEQ ID NO: 112 - DNA, 20 bp, Artificial Sequence
  Chemically synthesized primer sequence for Realtime-PCR XUT2-RTP-down
atccttggcgtcagattcag

SEQ ID NO: 113 - DNA, 21 bp, Artificial Sequence
  Chemically synthesized primer sequence for Realtime-PCR XUT3-RTP-up
ggcttcattggctagaaatcc

SEQ ID NO: 114 - DNA, 20 bp, Artificial Sequence
  Chemically synthesized primer sequence for Realtime-PCR XUT3-RTP-down
gcggaatattgctggaaatg

SEQ ID NO: 115 - DNA, 20 bp, Artificial Sequence
  Chemically synthesized primer sequence for Realtime-PCR XUT4-RTP-up
ggggacttttcgcaagtttc

SEQ ID NO: 116 - DNA, 21 bp, Artificial Sequence
  Chemically synthesized primer sequence for Realtime-PCR XUT4-RTP-down
ccctttgtctcaatccaacag

SEQ ID NO: 117 - DNA, 21 bp, Artificial Sequence
  Chemically synthesized primer sequence for Realtime-PCR XUT5-RTP-up
gtatcccgaaaccaagggtag

SEQ ID NO: 118 - DNA, 21 bp, Artificial Sequence
  Chemically synthesized primer sequence for Realtime-PCR XUT5-RTP-down
gtctctggcaatttggacaac

SEQ ID NO: 119 - DNA, 21 bp, Artificial Sequence
  Chemically synthesized primer sequence for Realtime-PCR XUT6-RTP-up
atggactctgtgttcgagagc

SEQ ID NO: 120 - DNA, 21 bp, Artificial Sequence
  Chemically synthesized primer sequence for Realtime-PCR XUT6-RTP-down
cgttctctaccctttggtacg

SEQ ID NO: 121 - DNA, 21 bp, Artificial Sequence
  Chemically synthesized primer sequence for Realtime-PCR XUT7-RTP-up
atactacgatgggcctcgttc

SEQ ID NO: 122 - DNA, 21 bp, Artificial Sequence
  Chemically synthesized primer sequence for Realtime-PCR XUT7-RTP-down
cgcccaaatctctagtttcag

SEQ ID NO: 123 - DNA, 21 bp, Artificial Sequence
  Chemically synthesized primer sequence for Realtime-PCR HGT1-RTP-up
gaagaagtcggacaaatgtgg

SEQ ID NO: 124 - DNA, 21 bp, Artificial Sequence
  Chemically synthesized primer sequence for Realtime-PCR HGT1-RTP-down
gtgggaaacatccatcttgtg

SEQ ID NO: 125 - DNA, 21 bp, Artificial Sequence
  Chemically synthesized primer sequence for Realtime-PCR HGT2-RTP-up
acgctagactcgaaacggaag

SEQ ID NO: 126 - DNA, 21 bp, Artificial Sequence
  Chemically synthesized primer sequence for Realtime-PCR HGT2-RTP-down
aggcaccaaaatagagtcgtc

SEQ ID NO: 127 - DNA, 21 bp, Artificial Sequence
  Chemically synthesized primer sequence for Realtime-PCR QUP1-RTP-up
cttgtttgaagttcccgcttg

SEQ ID NO: 128 - DNA, 21 bp, Artificial Sequence
  Chemically synthesized primer sequence for Realtime-PCR QUP1-RTP-down
cgctccttttctgaagaatcc

SEQ ID NO: 129 - DNA, 21 bp, Artificial Sequence
  Chemically synthesized primer sequence for Realtime-PCR QUP2-RTP-up
atgtctcttttctgggcttgg

SEQ ID NO: 130 - DNA, 21 bp, Artificial Sequence
  Chemically synthesized primer sequence for Realtime-PCR QUP2-RTP-down
caattcaggacccttcctacc

SEQ ID NO: 131 - DNA, 21 bp, Artificial Sequence
  Chemically synthesized primer sequence for Realtime-PCR QUP3-RTP-up
agggtgctatcttgtgtcctg

SEQ ID NO: 132 - DNA, 21 bp, Artificial Sequence
  Chemically synthesized primer sequence for Realtime-PCR QUP3-RTP-down
tttctgcctttcgaggtcttc

SEQ ID NO: 133 - DNA, 21 bp, Artificial Sequence
  Chemically synthesized primer sequence for Realtime-PCR QUP4-RTP-up
gtccttggaagaaatcgatgc

SEQ ID NO: 134 - DNA, 21 bp, Artificial Sequence
  Chemically synthesized primer sequence for Realtime-PCR QUP4-RTP-down
acgtattgcccaactgatctc

SEQ ID NO: 135 - DNA, 21 bp, Artificial Sequence
  Chemically synthesized primer sequence for Realtime-PCR LAC1-RTP-up
cgtggagaccaagaacttgac

SEQ ID NO: 136 - DNA, 21 bp, Artificial Sequence
  Chemically synthesized primer sequence for Realtime-PCR LAC1-RTP-down
agcaagcctgagcttctcttc

SEQ ID NO: 137 - DNA, 21 bp, Artificial Sequence
  Chemically synthesized primer sequence for Realtime-PCR LAC2-RTP-up
gacatgagaggcagaggaatg

SEQ ID NO: 138 - DNA, 21 bp, Artificial Sequence
  Chemically synthesized primer sequence for Realtime-PCR LAC2-RTP-down
ctggaaagcatcccaaaagac

SEQ ID NO: 139 - DNA, 21 bp, Artificial Sequence
  Chemically synthesized primer sequence for Realtime-PCR LAC3-RTP-up
acagaacctacgcagaattgg

SEQ ID NO: 140 - DNA, 20 bp, Artificial Sequence
  Chemically synthesized primer sequence for Realtime-PCR LAC3-RTP-down
catgctcgacaaattgcttg

SEQ ID NO: 141 - DNA, 21 bp, Artificial Sequence
  Chemically synthesized primer sequence for Realtime-PCR MAL1-RTP-up
gttccaagatggtgtttcagc

SEQ ID NO: 142 - DNA, 22 bp, Artificial Sequence
  Chemically synthesized primer sequence for Realtime-PCR MAL1-RTP-down
cagcattgtctacatggtcaac

SEQ ID NO: 143 - DNA, 21 bp, Artificial Sequence
  Chemically synthesized primer sequence for Realtime-PCR MAL2-RTP-up
agtgggtccaatgtgctactg

SEQ ID NO: 144 - DNA, 21 bp, Artificial Sequence
  Chemically synthesized primer sequence for Realtime-PCR MAL2-RTP-down
ttgggctcaacatgtaaggag

SEQ ID NO: 145 - DNA, 21 bp, Artificial Sequence
  Chemically synthesized primer sequence for Realtime-PCR MAL3-RTP-up
ttgttccaggacggtgttaag

SEQ ID NO: 146 - DNA, 22 bp, Artificial Sequence
  Chemically synthesized primer sequence for Realtime-PCR MAL3-RTP-down
cgttgttgtcatccttgttgtc

SEQ ID NO: 147 - DNA, 21 bp, Artificial Sequence
  Chemically synthesized primer sequence for Realtime-PCR MAL4-RTP-up
ttggtttgctacagcacttcc

SEQ ID NO: 148 - DNA, 21 bp, Artificial Sequence
  Chemically synthesized primer sequence for Realtime-PCR MAL4-RTP-down
ctagcgggaactcttttctcg

SEQ ID NO: 149 - DNA, 21 bp, Artificial Sequence
  Chemically synthesized primer sequence for Realtime-PCR MAL5-RTP-up
agaggattgggattggaagag

SEQ ID NO: 150 - DNA, 21 bp, Artificial Sequence
  Chemically synthesized primer sequence for Realtime-PCR MAL5-RTP-down
cctctttgttggtgattctgg

SEQ ID NO: 151 - DNA, 21 bp, Artificial Sequence
  Chemically synthesized primer sequence for Realtime-PCR MSF5-RTP-up
aaagacagccatggagagttg

SEQ ID NO: 152 - DNA, 21 bp, Artificial Sequence
  Chemically synthesized primer sequence for Realtime-PCR MSF5-RTP-down
tgcttccttgtcgaatgtacc

SEQ ID NO: 153 - DNA, 20 bp, Artificial Sequence
  Chemically synthesized primer sequence for Realtime-PCR AUT1-RTP-up
acgccaagtaccaaaccaag

SEQ ID NO: 154 - DNA, 21 bp, Artificial Sequence
  Chemically synthesized primer sequence for Realtime-PCR AUT1-RTP-down
gttggtgacagccaatctctg

SEQ ID NO: 155 - DNA, 21 bp, Artificial Sequence
  Chemically synthesized primer sequence for Realtime-PCR FUC1-RTP-up
ggatgggtatgagaggtttgg

SEQ ID NO: 156 - DNA, 21 bp, Artificial Sequence
  Chemically synthesized primer sequence for Realtime-PCR FUC1-RTP-down
aggaatcacagctgaaacacg

SEQ ID NO: 157 - DNA, 20 bp, Artificial Sequence
  Chemically synthesized primer sequence for Realtime-PCR ACT1-RTP-up
ccttggctccatcctctatg

SEQ ID NO: 158 - DNA, 21 bp, Artificial Sequence
  Chemically synthesized Primer sequence for Realtime-PCR ACT1-RTP-down
ccagattcgtcgtattcttgc

(19) United States
(12) Patent Application Publication
(10) Pub. No.: US 2012/0270290 A1
(43) Pub. Date: Oct. 25, 2012

(54) PENTOSE TRANSPORTER

(75) Inventors: Seiya Watanabe, Kyoto (JP); Shigeki Sawayama, Kyoto (JP)

(73) Assignee: KYOTO UNIVERSITY, Kyoto (JP)

(21) Appl. No.: 13/393,664

(22) PCT Filed: Sep. 1, 2010
(86) PCT No.: PCT/JP10/64962

§ 371 Date: Mar. 1, 2012

(30) Foreign Application Priority Data
Sep. 3, 2009 (JP) 2009-203362

(51) Int. Cl. C12P 7/10; C12P 7/06; C07K 14/00; C07H 21/04

(52) U.S. Cl. 435/165; 536/23.1; 435/161; 530/350

(57) ABSTRACT

Disclosed is the production of useful substances such as bioethanol from pentose through effective use of biomass resources. Specifically, the invention discloses the following: Use of HGT2 gene or an expression product protein thereof as a xylose transporter. A xylose and/or L-arabinose transporter as an expression product protein of a gene selected from the group consisting of HGT2 gene, XUT1 gene, and HXT2.4 gene. Use of a gene selected from the group consisting of HGT2 gene, XUT1 gene, and HXT2.4 gene as a xylose and/or L-arabinose transporter. A method for producing bioethanol, the method comprising introducing into yeast at least one gene selected from the group consisting of HGT2 gene, XUT1 gene, and HXT2.4 gene, and culturing the yeast in the presence of a biomass that contains xylose and/or L-arabinose.

### TECHNICAL FIELD

The present invention relates to membrane transport proteins (transporters) that promote uptake of arabinose and/or xylose.

### BACKGROUND ART

The ligneous biomass that accounts for a large proportion of the earth biomass is composed of lignocellulose. The makeup of a typical lignocellulose biomass is 35 to 45% cellulose, 25 to 40% hemicellulose, and 15 to 30% lignin. In recent years, cellulose and lignin have been actively studied to produce energy, as in the technique that converts these materials into glucose in a short time period using supercritical water. The hemicellulose can easily be decomposed to monosaccharides such as xylose by acid hydrolysis or enzyme degradation. Further, efficient conversion of xylose, a readily available sugar constituting a large percentage of hemicellulose, into liquid fuel is considered important from the standpoint of energy issues.

To date, there have been successes in the efficient anaerobic conversion of cellulose and other hexoses into ethanol with the use of many types of microorganisms such as yeast. However, it is known that efficient conversion of pentoses such as xylose is not possible.

Two main pathways are known for the xylose-to-ethanol conversion, as shown in FIG. 1. One is the single-step conversion of xylose into xylulose using xylose isomerase (XI), independent of a coenzyme. The other pathway involves the conversion of xylose into xylitol using xylose reductase (hereinafter, “XR”), and xylitol into xylulose using xylitol dehydrogenase (XDH). This pathway requires a coenzyme for the conversion. The both methods enable easy conversion of xylulose 5-phosphate produced by xylulose kinase (XK) into ethanol via the pentose.phosphate cycle, provided that xylulose is produced by conversion.

XI represents a xylose metabolic pathway commonly seen in bacteria. For example, bacteria such as Streptomyces sp. and Actinoplanes sp. are known to convert xylose into xylulose using XI, and then into ethanol via the pentose.phosphate cycle (Non-Patent Literature 1 and 2). However, the efficiency is very poor. The poor efficiency is believed to be due to the formation of organic acid as a by-product. The technique, therefore, has not been adapted to industrial applications. The Escherichia coli K011 strain developed in the United States represents an alternative technique in which two enzymes, pyruvate decarboxylase and alcohol dehydrogenase, of Zymomonas bacteria used for tequila production are expressed in E. coli (Patent Literature 1 and 2). This recombinant Escherichia coli is capable of producing ethanol by converting all the monosaccharides contained in a lignocellulose biomass. The technique, however, does not provide complete solutions to the formation of by-products, such as lactic acid, succinic acid, fumaric acid, and acetic acid, during the fermentation, and the low resistance to the fermentation inhibitor produced during the mashing of a lignocellulose biomass. Recent findings indicate the existence of XI in several species of eukaryotic microorganism rumen fungi.

The XR-XDH pathway is a pathway commonly found in eukaryotic microorganisms, and Pichia stipitis, Candida shehatae, and Pachysolen tannophilus are examples of eukaryotic microorganisms known to have this pathway (Non-Patent Literature 3 and 4). Though these eukaryotic microorganisms have been studied with a main focus on optimization of fermentation conditions, they are not the mainstream at present because of difficulties in controlling anaerobic and other conditions, and the poor resistance to alcohol and the degradation products that result from the mashing of the lignocellulose biomass. S. cerevisiae, on the other hand, is the most extensively studied eukaryotic microorganism for their potentially high alcohol fermentability and alcohol resistance.

However, because S. cerevisiae is incapable of utilizing xylose, the yeast is often used as an XR-XDH-XK genetically recombinant yeast by being genetically modified with the constitutively expressed P. stipitis-derived XR and XDH genes, and, in these days, three XK genes of S. cerevisiae. XI research has been slow because of the initial inability to functionally express this enzyme in yeast cells. Recently, there are successful attempts in XI expression and xylose fermentation using genes derived from rumen fungi; however, it is beginning to be understood that the efficiency is not as high as that of the XR-XDH system.

Broadly, there are two problems in the XR-XDH-XK genetically recombinant yeast, as has been pointed out from the beginning of development. The first problem is the accumulation of by-products such as xylitol, glycerol, and acetic acid during the fermentation process. One proposed reason for this is the cellular oxidation-reduction unbalance due to the different coenzyme requirements of XR and XDH in the catalytic reactions, and there have been attempts to improve the yeast coenzyme recycle system (Non-Patent Literature 5), and, in recent years, to artificially create an XR or XDH mutant of a different coenzyme specificity by site-specific mutation and improve xylose fermentation by using the mutant gene (Non-Patent Literature 6, 7, and 8).

The second problem is the extremely slow fermentation rate of the pentose compared to hexose. One reason for this slow fermentation rate is the delay in metabolism after the xylose→xylitol→xylulose→xylulose 5-phosphate conversion in a cell, and there have been attempts to make improvements by enhancing the expression of transketolase (TKL) and transaldolase (TAL) in the pentose.hosphate cycle other than XK (Non-Patent Literature 9). Another reason is the weak pentose transportability into cells compared to the hexose transport. The fact that xylose fermentation is enabled by the introduction of only XR-XDH or XI into Saccharomyces yeast indicates that the yeast inherently has xylose transportability (and transporters). Indeed, Hahn-Hagerdal et al. (Non-Patent Literature 10) and Ho et al. (Non-Patent Literature 11) have identified hexose transporters having xylose transportability, produced by individually introducing HXT1-7 and GAL2 to a mutant RE700 strain lacking both of the major hexose transporters HXT1-7 and GAL2. However, no improvement occurs in ethanol productivity even after these transporter genes are constitutively expressed.

Further, there have been attempts to introduce sugar transporter genes derived from other organisms. Hahn-Hagerdal et al. unsuccessfully attempted to simultaneously introduce to S. cerevisiae what appear to be xylose transporter genes derived from plants and bacteria, specifically xylose transporter genes from Escherichia coli (accession name, xylE), and chlorella (Hupl, accession No. X55349) and Arabidopsis (Stp2, accession No. NM_{—}100608; Stp3, accession No. AJ002399). It is difficult to cause yeast to functionally express genes derived from different organisms, and to accurately localize the genes in the membrane. Hahn-Hagerdal et al. does not make any analysis in this regard.

Hector et al. introduced Arabidopsis-derived xylose transporter genes (accession Nos. #BT015354, #BT015128) into S. cerevisiae having incorporated XR-XDH-XK gene in the chromosome (Non-Patent Literature 12). It has been confirmed by an immunofluorescent technique using a histidine tag added to amino acid terminals that the introduced genes were accurately expressed, and properly localized in the membrane. Effects on xylose fermentation were seen as 46%, 40%, and 70% improvements in xylose transportability, consumption rate, and ethanol productivity, respectively. Direct identification of a filamentous fungus Trichoderma reesei xylose transporter has been attempted by Ruohonen et al. (Non-Patent Literature 13). Ruohonen et al. constructed a library so as to express T. reesei cDNA in S. cerevisiae, introduced the library to a strain obtained by incorporating XR-XDH-XK gene into the chromosome of a S. cerevisiae mutant strain KY73 lacking a major hexose transporter, and screened for colonies that were able to grow by using xylose as a carbon source. Only a single colony was isolated, and Xlt1 was identified as a xylose transporter (accession No. AY818402). However, the result of retransforming a KY73 strain with this gene failed to confirm any growth on xylose or transportability. In the final analysis, the authors concluded that the growth ability on xylose after the Xlt1 introduction was acquired because of some natural mutation in the KY73 strain.

Goncalves et al. isolated xylose transporters from the cDNA library of xylose utilizing yeast Candida intermedia using two methods. GXF1 (accession No. AJ937350) was isolated by using a method that complements the phenotype of a S. cerevisiae hexose defective mutant. For GXS1 (accession No. AJ875406), cDNA was isolated from the partial amino acid sequence determined from the protein directly purified from the cell membrane of C. intermedia grown on xylose (Non-Patent Literature 14). Glucose and xylose transportability was observed in both GXF1 and GXS1. These genes were also expressed in an XR-XDH-XK genetically recombinant yeast for xylose fermentation. While GXF1 was functionally expressed in yeast cells, GXS1 was hardly transcribed nor was there any membrane localization (Non-Patent Literature 15). This indicates that sufficient expression in S. cerevisiae is not necessarily easy even with sugar transporter genes derived from a related yeast.

Even before the completion of the mapping of the genome sequence of xylose fermentative yeast Pichia stipitis in 2007, there had been some studies concerning sugar transport. Bisson et al. elucidated that two xylose transport systems, high-affinity (Kₘ=0.9 mM) and low affinity (Kₘ=380 mM), exist in P. stipitis, and that the latter is the same as the low-affinity glucose transport system (Non-Patent Literature 16). Weierstall et al. identified P. stipitis glucose transporters by taking advantage of complementarity to the S. cerevisiae RE700 strain (Non-Patent Literature 17). The first gene isolated was SUT1 gene (accession No. U77382), and the SUT1 was used as a probe in genomic southern hybridization to isolate SUT2 and SUT3 (accession Nos. AF0728080, U77581) having very similar nucleotide sequences. It is known that SUT1-3 is capable of transporting xylose in addition to hexose, and, kinetically, SUT1 is most desirable. Kondo et al. introduced SUT1 in the form of a plasmid into a Saccharomyces yeast that had incorporated XR-XDH-XK gene in the chromosome (Non-Patent Literature 18). While this improved the xylose consumption rate in xylose fermentation, the final ethanol yield remained the same. This study is the only example that can be said as a successful attempt using the sugar transporter gene introducing technique in the present field.

Nevertheless, the xylose consumption rate in the example of Kondo et al. is still at least 6 times slower than that for glucose, and is far from practical applications. Even though the study demonstrated some effectiveness of the P. stipitis SUT1, the gene was identified as a glucose transporter in the first place, and may not represent the most competent xylose transporter of P. stipitis. In fact, SUT1 expression is not induced at all in the presence of xylose in both aerobic and anaerobic conditions, and expression is higher in SUT2 and SUT3 having poorer xylose transportability (Non-Patent Literature 17).

Even though L-arabinose is not abundant in wood biomass, this pentose accounts for 15% of the constituent components in, for example, corn stover (stem), and is considered as a major pentose in a lignocellulose biomass along with xylose. Because bacteria have a metabolic pathway that uses L-arabinose isomerase (AraA), ribulokinase (AraB), and ribulose-5-phosphate 4-isomerase (AraD), L-arabinose fermentation becomes possible when given fermentability as in Escherichia coli KO11 (FIG. 2). Partly because the metabolic pathways of eukaryotic microorganisms were not fully understood, attempts for L-arabinose fermentation in S. cerevisiae were made by introducing the pathways of these bacteria (mainly Escherichia coli and Bacillus subtilis), only to be encountered with initial difficulties as these enzymes were hardly expressed in the yeast cells (Non-Patent Literature 19). Reasonable expression levels were ensured by optimizing the codons of the introduced genes for yeast (Non-Patent Literature 20). Further, because the GAL2 of S. cerevisiae is capable of L-arabinose transport, overexpression under the control of a constitutive expression promoter was attempted. While these improvements enabled L-arabinose fermentation, it was always accompanied by a long period of acclimation using subcultures.

Penttila et al. in a series of studies identified all L-arabinose metabolism genes of eukaryotic microorganisms (Non-Patent Literature 21 and 22). According to the authors, L-arabinose conversion occurs from L-arabinitol→L-xylulose→xylitol→xylulose→xylulose 5-phosphate (FIG. 2). Because the first reductase and the last two enzymes are XR, XDH, and XK, respectively, it is reasoned that the only new enzymes over the xylose metabolic pathway are L-arabinitol 4-dehydrogenase (LADH) and L-xylulose reductase (LXR). LADH and LXR genes were thus expressed in an XR-XDH-XK gene recombinant S. cerevisiae (Non-Patent Literature 23). In contrast to the bacterial genes, no problem was presented in the expression in yeast cells. However, there was essentially no ethanol production, and L-arabinitol accumulated as a by-product.

There is no study concerning the L-arabinose transporters of eukaryotic microorganisms that can specifically metabolize L-arabinose. This is probably because the overexpression of S. cerevisiae GAL2 is effective to some extent. Knoshaug et al. conducted studies of L-arabinose transport in two yeasts, Kluyveromyces maxianus and Pichia guilliermondii, that grow well on L-arabinose (Patent Literature 3). KmLAT1 and PgLAT2 genes believed to be L-arabinose transporters were identified from these yeasts, and introduced as a plasmid to a recombinant Saccharomyces yeast overexpressing bacterial AraA, AraB, and AraD. As a result, coexpression of S. cerevisiae GAL2 and PgLAT2 increased the growth doubling time with the L-arabinose used as a carbon source. Ethanol production appears to be absent. Hahn-Hagerdal et al. identified L-arabinose transporter genes from L-arabinose metabolizing yeast Candida arabinoferrnentas (Patent Literature 4). No fermentation experiment was conducted using S. cerevisiae.

The P. stipitis genome actually includes another similar homolog, named SUT4, aside from SUT2 and SUT3. The SUT1-4 genes are closest to the hexose transporters HXT of Saccharomyces yeast amongst a little less than 40 (estimate) sugar transporters of P. stipitis in a molecular phylogenetic tree created from the amino acid sequences of known eukaryotic microorganism sugar transporters (FIG. 3). Thus, it can be anticipated to some degree that these are capable of glucose and xylose transport. On the other hand, as to the substrate specificity of the known eukaryotic microorganism sugar transporters, it can be seen from the phylogenetic tree that there is absolutely no link between the relatedness and sugar transportability except for only a few sugar transporters that can be estimated as maltose or lactose transporters. Specifically, there is currently no means of knowing any more information concerning other non-SUT (estimated) sugar transporters of P. stipitis.

### CITATION LIST

### Patent Literature

PTL 1: U.S. Pat. No. 5,000,000
PTL 2: U.S. Pat. No. 5,821,093
PTL 3: WO2007/143247
PTL 4: WO2009/008756

### Non-Patent Literature

NPL 1: Ladisch, M. R. et al. (1983) Enzyme Microb. Technol., 5: 82-102
NPL 2: Skoog, K. et al. (1988) Enzyme Microb. Technol., 10: 66-78
NPL 3: Slininger, P. J. et al. (1987) Enzyme Microb. Technol. 9: 5-15
NPL 4: Prior, B. A. et al. (1989) Proc. Biochem., 2: 21-32
NPL 5: Olsson, L. et. al. (2003) Appl. Environ. Microbiol. 69: 4732-4736.
NPL 6: Watanabe, S. et al. (2005) J. Biol. Chem. 280: 10340-10349.
NPL 7: Watanabe, S. et al. (2007) J. Biotechnol. 130, 316-319.
NPL 8: Watanabe, S. et al. (2007) Microbiology 153, 3044-3054.
NPL 9: Hahn-Hagerdal B. et al. (1995) Appl. Environ. Microbiol. 1995, 61, 4184-4190.
NPL 10: Hahn-Hagerdal B. et al. (2002) Microbiology 148, 2783-2788
NPL 11: Sedlak, M. and Ho, N. W. Y. (2004) Yeast 21, 671-684.
NPL 12: Hector, R. E. et al. (2008) Appl Microbiol Biotechnol 80, 675-684
NPL 13: Ruohonen, L. et al. (2007) Appl Microbiol Biotechnol 74: 1041-1052
NPL 14: Goncalves, P. et al. (2006) Biochem. J. 395, 543-549.
NPL 15: Goncalves, P. et al. (2008) Microbiology 154, 1646-1655
NPL 16: Does, A. L. and Bisson, L. F. (1989) Appl. Environ. Microbiol. 55, 159-164.
NPL 17: Weierstall, T. et al. (1999) Mol. Microbiol. 31, 871-83.
NPL 18: Kondo, A. et al. (2008) Enzyme Microb. Technol. 43, 115-119.
NPL 19: Sedlak, M. and Ho, N. W. Y. (2001) Enzyme Microb. Technol. 28, 16-24.
NPL 20: Wiedemann, B. and Boles, E. (2008) Appl. Environ. Microbiol. 74, 2043-2050.
NPL 21: Richard, P. et al. (2001) J. Biol. Chem. 276, 40631-40637.
NPL 22: Richard, P. et al. (2002) Biochemistry 41, 6432-6437.
NPL 23: Richard, P. et al. (2003) FEMS Yeast Res. 3, 185-189.

### SUMMARY OF INVENTION

### Technical Problem

It is an object of the present invention to provide a solution to the energy issues by providing a means to convert to ethanol at high efficiency from monosaccharides that can be easily obtained by the hydrolysis of hemicellulose that accounts for about 30% or more of a biomass resource such as wood.

### Solution to Problem

The present inventors constructed a system to express all the (estimated) sugar transporter genes of P. stipitis in Saccharomyces yeast, and tried to find pentose transporters important in the yeast breeding development for bioethanol production through the comprehensive analyses of the substrate specificity of individual genes. As a result, the inventors found that specific genes can function as pentose transport proteins in yeast.

The present invention provides the genes below or expression product proteins thereof, i.e., transporters of pentoses such as xylose and arabinose, use thereof, and a method for producing bioethanol.

### Item 1:

Use of HGT2 gene or an expression product protein thereof as a xylose transporter.

### Item 2:

A xylose and/or L-arabinose transporter as an expression product protein of a gene selected from the group consisting of HGT2 gene, XUT1 gene, and HXT2.4 gene.

### Item 3:

Use of a gene selected from the group consisting of HGT2 gene, XUT1 gene, and HXT2.4 gene as a xylose and/or L-arabinose transporter.

### Item 4:

A method for producing bioethanol, the method comprising introducing into yeast at least one gene selected from the group consisting of HGT2 gene, XUT1 gene, and HXT2.4 gene, and culturing the yeast in the presence of a biomass that contains xylose and/or L-arabinose.

### Item 5:

A bioethanol producing method according to Item 4, wherein the biomass containing xylose and/or L-arabinose is a lignocellulose.

### Advantageous Effects of Invention

The present invention can promote use of pentoses by greatly enhancing the pentose uptake performance of microorganisms having poor uptake of pentoses such as xylose and arabinose. This is achieved by introducing genes into the microorganisms.

Further, by simultaneously introducing enzymes' associated with pentose metabolism to a microorganism such as yeast, ethanol can be efficiently produced from a ligneous biomass of lignocellulose.

Non-Patent Literature 18 fails to improve bioethanol yield even by introduction of SUT1 into the host S. cerevisiae MT8-1 that has incorporated XR-XDH-XK in the chromosome. The present application, on the other hand, demonstrated improved ethanol yield by introduction of SUT1 gene into the same host. In contrast to Kondo et al. (Non-Patent Literature 18) that directly adds all deficient amino acids into a culture medium for transformation, the present invention complements auxotrophy by addition of a plasmid, except for adenine. Though not to be bound by theory, it is thus considered desirable to complement auxotrophy by addition of a plasmid.

### BRIEF DESCRIPTION OF DRAWINGS

FIG. 1 is a diagram representing a xylose metabolic pathway.

FIG. 2 is a diagram representing L-arabinose metabolic pathways of (A) bacteria and (B) eukaryotic microorganism.

FIG. 3 is a diagram representing a molecular phylogenetic tree of eukaryotic microorganism sugar transporters. Bold face indicates the P. stipitis sugar transporters analyzed. Functional analyses of all the sugar transporters of other organisms have been completed. Sc, Saccharomyces cerevisiae; Kl, Kluyveromyces lactis; Td, Torulaspora delbrueckii; Ci, Candida intermedia; Sp, Schizosaccharomyces pombe; Am, Amanita muscaria; Uf, Uromyces fabae; An, Aspergillus niger; Hp, Hansenula polymorpha; Cn, Cryptococcus neoformans; Dh, Debaryomyces hansenii; Bc, Botrytis cinerea; Tr, Trichoderma reesei; Ca, Candida albicans.

FIG. 4 is a diagram showing the primer sequences used for the subcloning of P. stipitis (estimated) sugar transporter genes. Superscript a: the internal HindIII site (double underlined) is abolished. Subscript b: SUT2-4 occur almost at the same nucleotide site, and cannot be isolated by simple genomic PCR. Genomic fragments containing SUT2-4 were thus amplified with SUT2-up+SUT2-down, SUT3-up+SUT3-down, and SUT4-up+SUT4-down primers, respectively, and the genes were amplified with their respective primers using these fragments as templates. Superscript c: the internal EcoRI site (double underlined) is abolished. Superscript d: cDNA was synthesized by two runs of PCR for genes (HGT1, LAC3, QUP3, QUP4) containing introns. Superscript e: the internal HindIII site (double underlined) is abolished.

FIG. 5 is a diagram representing the PCR results of subcloning P. stipitis (estimated) sugar transporter genes by using the cDNA incorporated in a pPGK plasmid as a template. Numbers in parentheses indicate gene base length.

FIG. 6 is a diagram representing the strains and plasmids used in the present study.

FIG. 7 is a diagram representing the substrate specificity of P. stipitis (estimated) sugar transporter genes. S. cerevisiae KY73 strains were transformed with the genes, and grown on YNB minimal media that contained (A) glucose, (B) D-mannose, (C) fructose, and (D) galactose as the carbon source. Genes that showed no growth are not presented. The vertical axis in the graphs represents OD600 value.

FIG. 8 is a diagram identifying P. stipitis (estimated) sugar transporter genes having xylose transportability (A). S. cerevisiae KY73 strains expressing the genes were cultured with maltose, and the presence or absence of xylose in the cells was analyzed by HPLC after adding xylose. Genes having transportability are presented in (B).

FIG. 9 is a diagram representing the time-course xylose transport by P. stipitis xylose transporters. The results are based on the analysis of the yeasts sampled every 30 minutes after the addition of xylose in the experiment of FIG. 8. Numerical values are presented as the sum of xylose and xylitol amounts. Insert graphs represent data for xylose and xylitol.

FIG. 10 is a schematic view of a pAUR-XR-XDH-XK plasmid. P. stipitis-derived XR gene and XDH gene, and S. cerevisiae-derived XK gene were expressed using PGK promoters, and homologously recombined on the S. cerevisiae chromosome by cutting the BsiWI restriction enzyme site of aureobasidin resistant gene AUR1-C for transformation.

FIG. 11 is a diagram representing xylose fermentation by P. stipitis xylose transporters in Saccharomyces yeast. Each gene was introduced as a plasmid into a S. cerevisiae KY73-XYL strain that had xylose metabolizing enzyme but did not have transportability. The vertical axis represents component concentration g/L, and the horizontal axis represents fermentation time h. (A) xylose concentration, (B) xylitol concentration, (C) glycerol concentration, (D) ethanol concentration.

FIG. 12 is a diagram representing xylose fermentation by P. stipitis xylose transporters in Saccharomyces yeast. Each gene was introduced as a plasmid into an MT8-1-XYL strain obtained by conferring the xylose utilizing ability to a wild-type Saccharomyces yeast having normal hexose transporters. (A) xylose concentration, (B) ethanol concentration. Minute amounts of xylose and acetic acid were detected as by-products (data not shown).

FIG. 13 is a diagram identifying P. stipitis (estimated) sugar transporter genes having L-arabinose transportability (A). S. cerevisiae KY73 strains expressing the genes were cultured with maltose, and the presence or absence of xylose in the cells was analyzed by HPLC after adding xylose. Genes having transportability are presented in (B).

FIG. 14 is a diagram representing the time-course L-arabinose transport by P. stipitis L-arabinose transporters. The results are based on the analysis of the yeasts sampled every 30 minutes after the addition of L-arabinose in the experiment of FIG. 13.

FIG. 15 is a diagram representing the primer sequences used for real-time PCR analysis. The target genes of real-time PCR analysis are presented under the column headed “Gene”.

FIG. 16 is a diagram showing genes whose expression is induced when P. stipitis is cultured on minimal media containing specific carbon sources. The carbon sources contained in the media used in the experiments are (A) glucose, (B) mannose, (C) fructose, (D) galactose, (E) xylose, and (F) L-arabinose. The component series in (A) to (F) represent, from the left, the expression levels of RGT2, SNF3, SUT1, SUT2/3/4, HXT2.1, HXT2.2, HXT2.3, HXT2.4, HXT2.5/2.6, HXT4, XUT1, XUT2, XUT3, XUT4, XUT5, XUT6, XUT7, HGT1, HGT2, QUP1, QUP2, QUP3, QUP4, LAC1, LAC2, LAC3, MAL1, MAL2, MAL3, MAL4, MAL5, MFS5, STL1, AUT1, and FUC1 genes, as shown in (G).

FIG. 17 is a diagram showing genes whose expression is induced when P. stipitis is cultured on minimal media containing specific carbon sources. The expression levels are for (A) RGT2 gene, (B) SUT1 gene, (C) SUT2/3/4 gene, (D) HXT2.4 gene, (E) XUT1 gene, and (F) HGT2 gene. The component series in (A) to (F) represent the experiment results from media containing, from the left, glucose, mannose, fructose, galactose, xylose, L-arabinose, D-arabinose, L-rhamnose, maltose, cellobiose, sucrose, lactose, and glycerol, as shown in (G).

FIG. 18 is a diagram representing the sugar transportability of HGT2 and XUT1. (A) xylose transportability; (B), L-arabinose transportability. The open circle indicates sugar transportability when only HGT2 was expressed in the presence of xylose alone. The open triangle indicates sugar transportability when only HGT2 was expressed in the presence of xylose and L-arabinose. The open diamond indicates sugar transportability when HGT2 and XUT1 were expressed in the presence of xylose and L-arabinose.

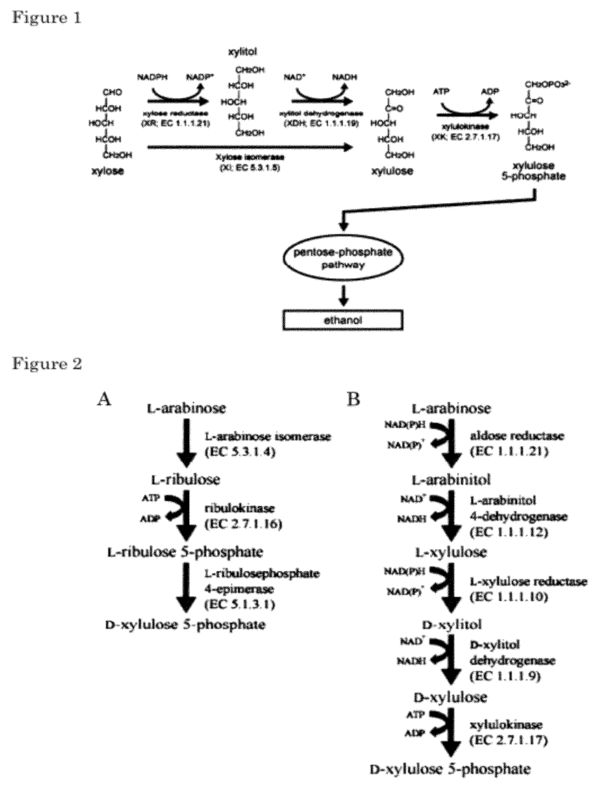

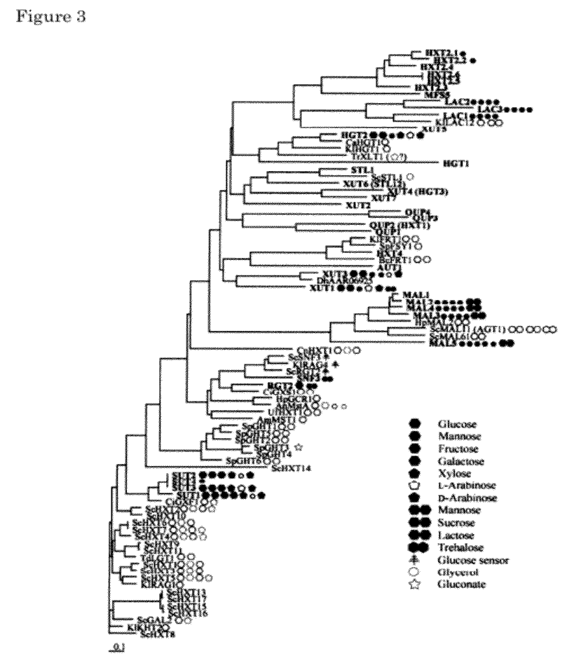

### DESCRIPTION OF EMBODIMENTS

In the specification, the HGT2 gene or an expression product protein thereof is preferably one derived from P. stipitis (NCBI accession No. XM_{—}001382718). Other examples include a wide range of other eukaryotic cell- or prokaryotic cell-derived HGT2 genes or expression product proteins thereof, either modified or unmodified, having functions as a xylose transporter and/or an L-arabinose transporter.

In the specification, the SUT1 gene or an expression product protein thereof is preferably one derived from P. stipitis (NCBI accession No. U77382). Other examples include a wide range of other eukaryotic cell- or prokaryotic cell-derived SUT1 genes or expression product proteins thereof, either modified or unmodified, having functions as a xylose transporter and/or an L-arabinose transporter.

In the specification, the XUT1 gene or an expression product protein thereof is preferably one derived from P. stipitis (NCBI accession No. XM_{—}001385546). Other examples include a wide range of other eukaryotic cell- or prokaryotic cell-derived XUT1 genes or expression product proteins thereof, either modified or unmodified, having functions as a xylose transporter and/or an L-arabinose transporter.

In the specification, the HXT2.4 gene or an expression product protein thereof is preferably one derived from P. stipitis (NCBI accession No. XM_{—}001387720). Other examples include a wide range of other eukaryotic cell- or prokaryotic cell-derived HXT2.4 genes or expression product proteins thereof, either modified or unmodified, having functions as a xylose transporter and/or an L-arabinose transporter.

In the specification, the genes including HGT2, XUT1, SUT1, and HXT2.4 are incorporated either alone or in combination into a host capable of expressing the genes, and the proteins having the amino acid sequences encoded by the genes are used as xylose transporters and/or L-arabinose transporters. Known conditions commonly used for the expression of a foreign gene incorporated in a host cell may be used for the expression.

In the specification, the genes including HGT2, XUT1, SUT1, and HXT2.4 are incorporated either alone or in combination into a host capable of ethanol fermentation, and bioethanol can be produced by fermenting the host in the presence of a biomass that contains xylose and/or L-arabinose. Fermentation is performed under the same conditions used for a host that has not incorporated the genes, and known conditions may be used.

The biomass used for the ethanol fermentation is not particularly limited, as long as it contains xylose and/or arabinose, and a wide range of known biomasses may be used. Examples of the biomass include lignocellulose-containing wood, rice straw, and waterweed.

### EXAMPLES

The present invention is described below in detail based on examples. It should be noted that the present invention is in no way limited by the following examples.

### Example 1

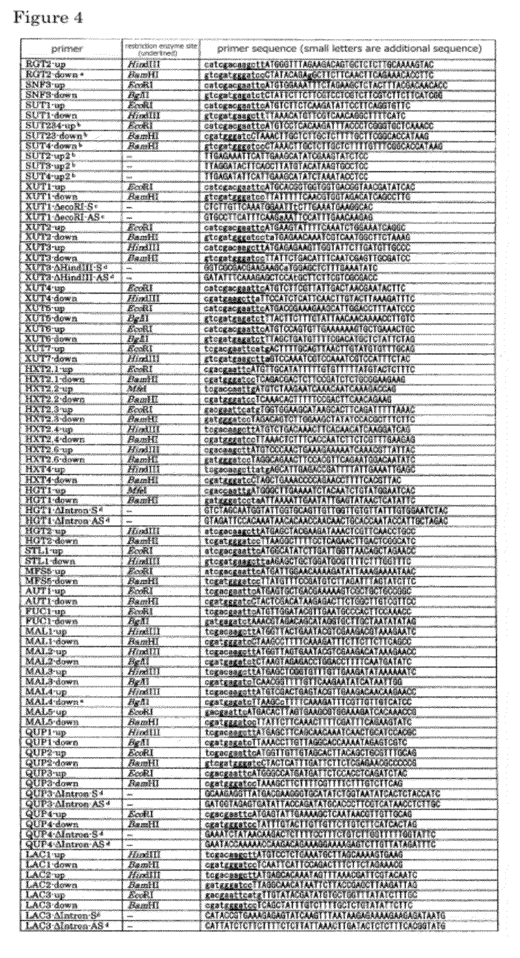

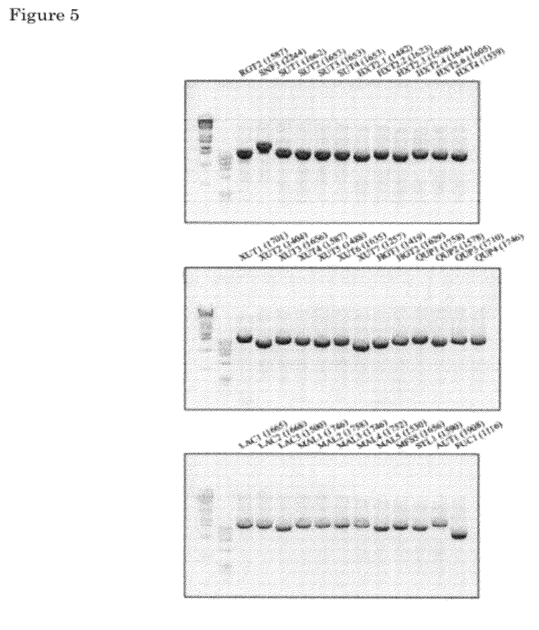

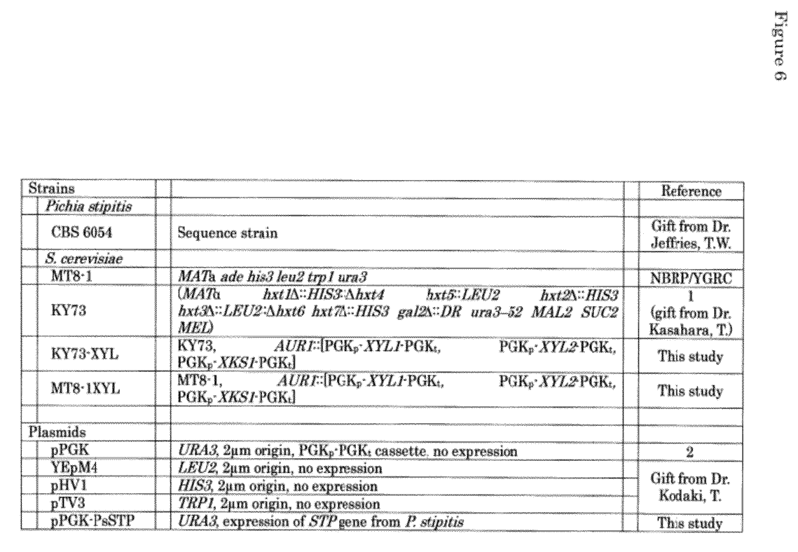

Using the Saccharomyces yeast hexose transporter (HXT) as a probe, a Protein-Blast search was performed against the P. stipitis genome sequence, and 38 homologous genes were screened for with the lower limit of about 30%. Oligonucleotide primers were designed so that appropriate restriction enzyme sites were added to the 5′- and 3′-ends of each gene (FIG. 4). The cDNA after the restriction enzyme treatment was subcloned into a pPGK plasmid that included a phosphoglycerate kinase (PGK) promoter.terminator cassette (the PGK promoter being a constitutive expression promoter of Saccharomyces yeast) and the marker gene URA3. FIG. 5 represents the results of the PCR experiments that used each plasmid as a template.

In order to examine the transportability for different hexoses, specifically glucose, D-mannose, D-fructose, and D-galactose, a Saccharomyces yeast mutant strain KY73 lacking both of the major hexose transporters HXT1-7 and GAL2 was used. This strain cannot grow on carbon sources other than maltose, and is auxotrophic only for uracil. The KY73 strain was transformed with a pPGK plasmid containing the P. stipitis sugar transporter gene, and was screened on an YNB medium plate that contained maltose as the carbon source. Single colonies were selected, and the presence or absence of growth on YNB liquid media that contained glucose, D-mannose, D-fructose, and D-galactose as the carbon source was observed. As a result, the following genes were identified as having transportability for the hexoses.

### Glucose: SUT1, HGT2, SUT3, SUT2, XUT3, SUT4, XUT1, HXT2.2

### D-mannose: SUT1, SUT2, SUT3, SUT4, HGT2, RGT2, XUT3, XUT1

### D-fructose: SUT1, SUT2, XUT3, SUT3, SUT4, HGT2

### D-galactose: SUT1, SUT3

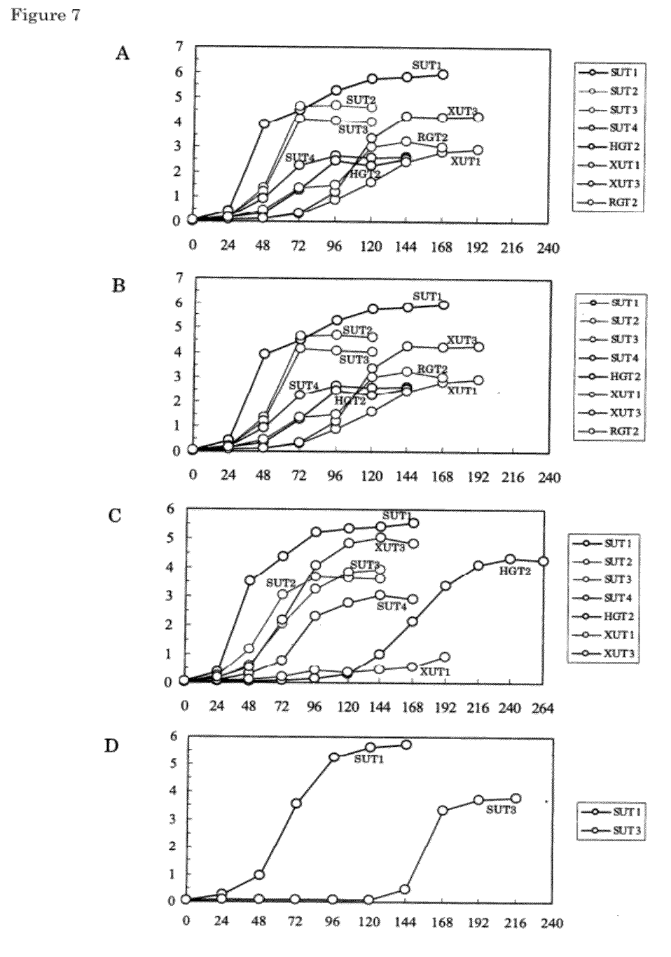

The growth curve is shown in FIG. 7. It can be seen that hexose transportability, overall, is observed only in the limited sugar transporter genes screened, and that these genes are capable of simultaneously transporting glucose, D-mannose, and D-fructose, but not D-galactose. In order to identify P. stipitis glucose transporter genes, Weierstall et al. introduced the genomic library of P. stipitis into a Saccharomyces yeast mutant strain Y having the same mutation as KY73 but different from KY73. In this way, Weierstall et al. identified SUT1. The homologs SUT2 and SUT3 were also identified. This result coincides well with the result of the present experiment in which SUT1-3 most strongly complemented the KY73 hexose transportability.

### Example 2

### D-xylose Transporter

### (1) Screening of Xylose Transporter Genes

Experiments were conducted to identify transporters for D-xylose, a major pentose in a lignocellulose biomass, as follows. A KY73 strain carrying a pPGK plasmid that includes a P. stipitis sugar transporter gene was cultured at 30° C. for 3 days in 10-mL YNBMal (minimal medium containing maltose as the carbon source). All maltose in the medium is consumed in this process. This was followed by addition of 400 μL of a 50% D-xylose solution (final concentration 2%). After being agitated for 2 hours, the yeast cells were collected by centrifugation, and washed twice with ice-cooled sterile water (30 mL). The collected yeast cells were suspended in 400-μL sterile water, and agitated at 37° C. and 200 rpm for 1 hour. As a result, the D-xylose transported into the cells by the introduced gene discharged from the cells. The D-xylose concentration in the supernatant was identified by differential refractometry (RI) using a HPLC system attached to an Aminex HPX-87H column.

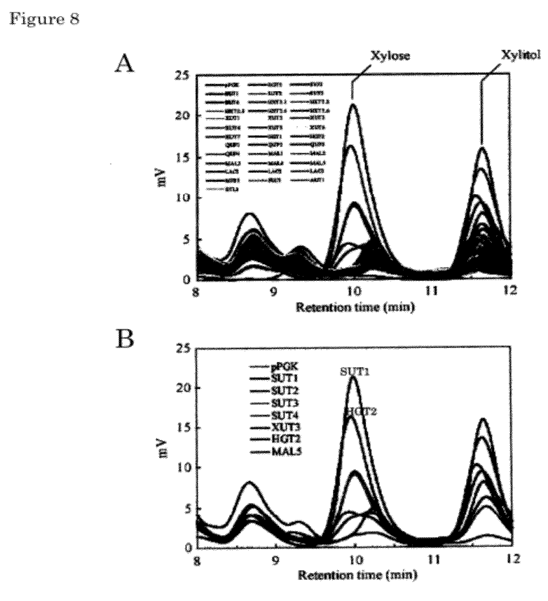

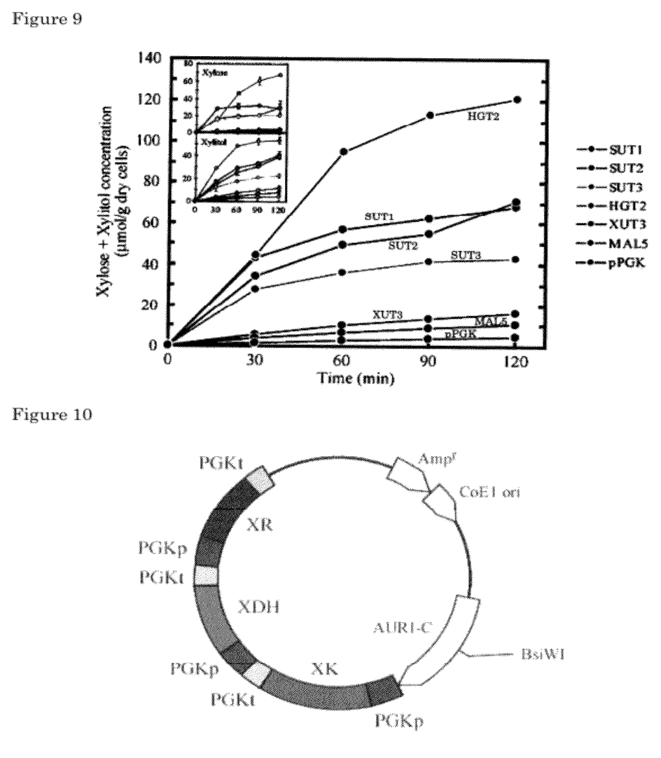

The HPLC elution curves are presented in FIG. 8. Xylose transport into the cells was confirmed in SUT1, HGT2, SUT2, SUT3, MAL5, XUT3, and SUT4. The elution curves showed peaks (11.7 min) that appeared to be of xylitol, in addition to peaks (10 min) corresponding to xylose. This is believed to be due to the xylitol production from xylose by GRE3, an XR homolog of Saccharomyces yeast. The xylose transport was measured over a time course for the seven genes (FIG. 9). Xylose transport was most active in HGT2, and the xylitol level was the highest also in this gene, followed by SUT1, SUT2, and SUT3. The xylose transportability of these genes has been elucidated by the previous studies of Weierstall et al.

### (2) Xylose Fermentability of Xylose Transporters

Experiments were conducted to examine the properties of the major xylose transporters HGT2, SUT1, SUT2, and SUT3 in xylose fermentation. First, a plasmid pAUR-XR-XDH-XK for incorporation into the yeast chromosome, including a cassette containing P. stipitis-derived XR and XDH genes and a Saccharomyces yeast-derived XK gene each ligated to a PGK promoter was introduced to a KY73 strain (FIG. 10). The plasmid also carries a resistant gene AUR1-C against aureobasidin that has an antimicrobial effect against eukaryotic microorganisms. The XR-XDH-XK gene can thus be stably introduced into the chromosome by the homologous recombination with the allele AUR located on the Saccharomyces yeast chromosome, using the aureobasidin resistance as a marker. The strain so produced was named KY73-XYL.

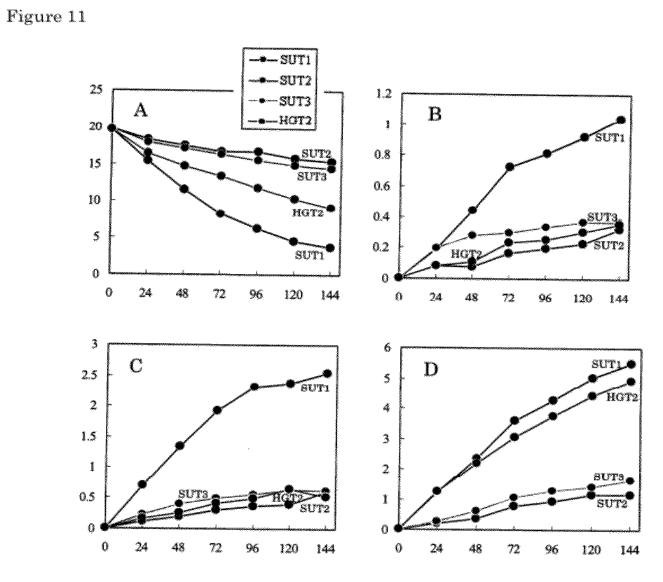

The KY73-XYL was then transformed with pPGK plasmids carrying HGT2, SUT1, SUT2, and SUT3. The xylose incorporated into the cells by these genes is metabolized to ethanol. The KY73-XYL strain can be grown using xylose as the only carbon source, and a fermentation experiment was conducted with a 200-mL baffled flask at a rotation speed of 150 rpm, using 20 g/L xylose as the carbon source (FIG. 11). It can be seen from the xylose consumption rate and the product ethanol concentration that xylose fermentation was sufficient in SUT1 and HGT2. By comparing SUT1 and HGT2, xylose consumption was about twice as fast in SUT1 than in HGT2. However, the amounts of the product ethanol after 6 days were not greatly different from HGT2, which produced about 90% of the ethanol produced in SUT1 after the 6-day period. This is believed to be due to the greater accumulation of xylitol (about 2.9 times) and glycerol (about 4.9 times) in SUT1 than in HGT2, and the resulting failure to efficiently convert the incorporated xylose into ethanol.

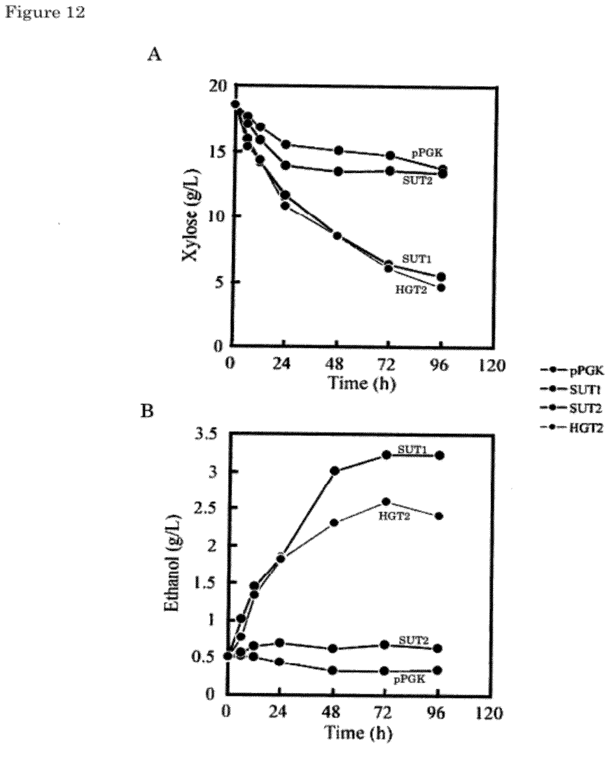

Xylose fermentation was performed using a Saccharomyces yeast that had an intact hexose transporter, instead of the KY73 strain. First, the pAUR-XR-XDH-XK plasmid was introduced to the host yeast MT8-1 strain (MATa ade his3 leu2 trp1 ura3; named MT8-1XYL). Genomic PCR and the enzyme activity in the acellular extract confirmed proper incorporation of the XR and XDH genes, and increased XK gene activity. The MT8-1XYL strain was then transformed with pPGK, pPGK-SUT1, pPGK-SUT2, and pPGK-HGT2 (all ura3+) plasmids on YNB plates to which YEpM4 (leu2+), pHV1 (his3+), and pTV3 (trp1+) had been applied together with adenine (the strains have the same names as the plasmids). The transformed yeasts were cultured in an adenine-containing YNB minimal medium using glucose as the carbon source, and used in a fermentation experiment conducted with a 200-mL baffled flask at a rotation speed of 150 rpm using adenine-containing 20 g/L xylose as the carbon source. The results are presented in FIG. 12.

Xylose consumption was notably slow in the control pPGK, leaving 13.7 g/L after 4 days. No ethanol production was confirmed either. On the other hand, SUT1 and HGT2 had xylose consumption 2.7 times and 2.9 times, respectively, higher than that of pPGK after 4 days, and produced 3.2 g/L and 2.4 g/L, respectively, of ethanol. Some improvement was seen in SUT2; however, the rate was much lower than in SUT1 and HGT2. These results coincide well with the results of the xylose fermentation experiments performed for the individual genes using KY73-XYL.

### Example 3

### L-Arabinose Transporter

Experiments were conducted to identify transporters for L-arabinose, another major pentose in a lignocellulose biomass along with D-xylose, as follows. Incorporation of the P. stipitis sugar transporter genes into cells using the KY73 strain was measured according to the methods used for D-xylose.

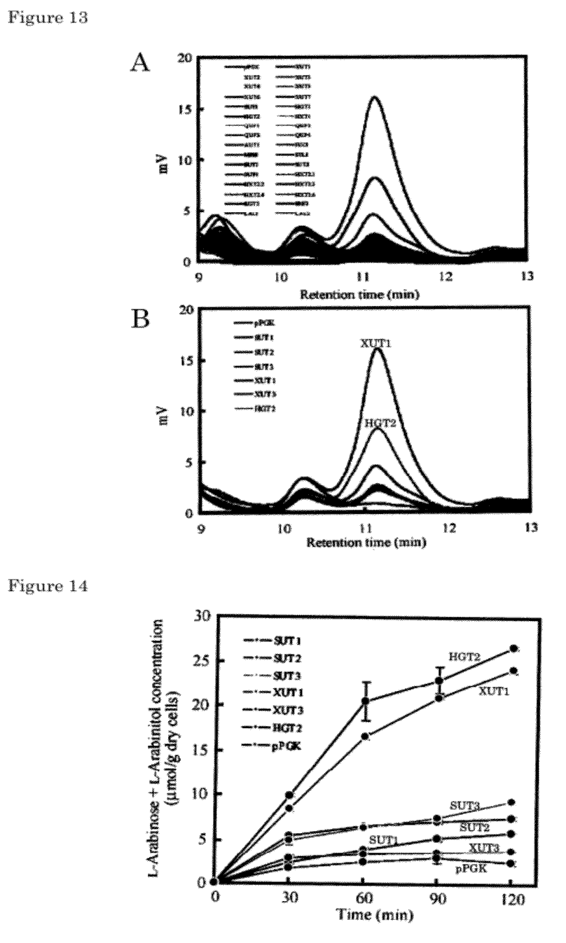

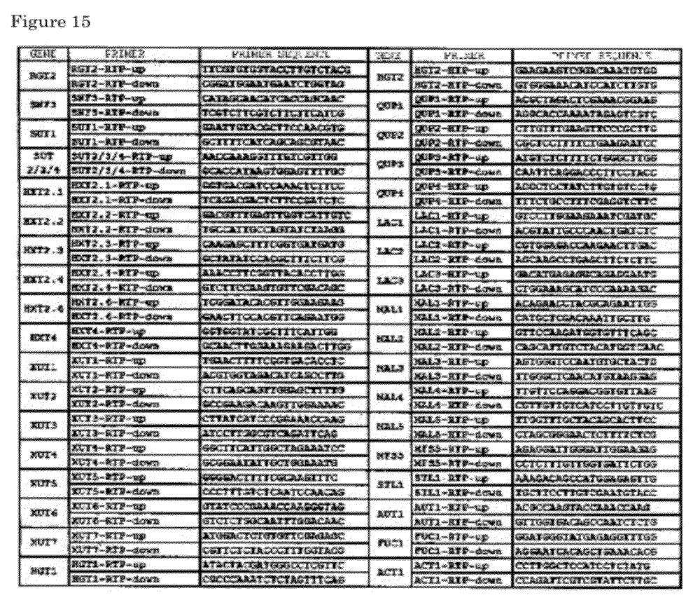

FIG. 13 represents the results of the HPLC measurements of the L-arabinose concentration within the cells. Whilst the L-arabinose peak occurs around 10.8 min, the identified peaks were around 11.2 min, a position corresponding to L-arabinitol. This is probably because of the action of the Saccharomyces yeast GRE3 serving as L-arabinose reductase and converting the L-arabinose incorporated in the cells into L-arabinitol. The L-arabinitol peaks were confirmed in the P. stipitis sugar transporter genes XUT1, HGT2, SUT3, SUT2, SUT1, and XUT3. The L-arabinose transport was measured over a time course for the seven genes (FIG. 14). The highest L-arabinose transport was confirmed in HGT2 and XUT1.

### Example 4

### Gene Expression Analysis by Real-Time PCR Method

The foregoing descriptions were directed to the analyses of the sugar transportability of the translated products of the sugar transporter genes using primarily the Saccharomyces yeast mutant strain KY73. However, for example, just because sugar transporter gene A has the ability to transport sugar B does not necessarily mean that the gene is functional in the metabolism of sugar B in P. stipitis. Strictly speaking, phenotyping of mutant strains produced by individually destroying the sugar transporter genes is necessary. However, in this experiment, the correlation between protein functions and gene expression was comparatively examined by estimating the mRNA levels of the sugar transporter genes by real-time PCR after culturing P. stipitis in each different minimal medium containing each sugar as the carbon source, based on the common knowledge that the expression of a transporter gene that uses a certain sugar as the substrate is induced under growth in which the same sugar is used as the carbon source.

The amplification primers for the real-time PCR were designed using the Primer3 program (http://frodo.wi.mit.edu/primer3/input.htm), and 100 to 150 bp from the gene of interest were amplified. The specific sequences of the primers used in this example are shown in FIG. 15. The P. stipitis was aerobically cultured on minimal media containing glucose, mannose, fructose, galactose, xylose, L-arabinose, D-arabinose, L-rhamnose, maltose, cellobiose, sucrose, lactose, and glycerol as the carbon source each in 2% (w/v) concentration. The bacteria were collected in the logarithmic growth phase (OD600=0.6 to 0.8), and RNA was extracted using an RNeasy® Mini Kit (Qiagen). The extracted RNA (100 ng) was then used to perform cDNA reverse-transcription using a PrimerScript® RT reagent Kit (Perfect RealTime; TaKaRa). Real-time PCR analysis was performed using a SYBR® Premix Ex Taq™ (Perfect Real Time; Bio-Rad). Actin gene (ACT1) was used as a house-keeping gene.

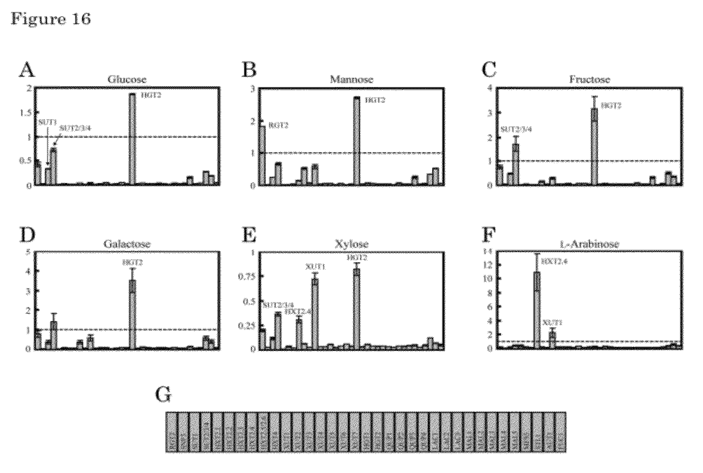

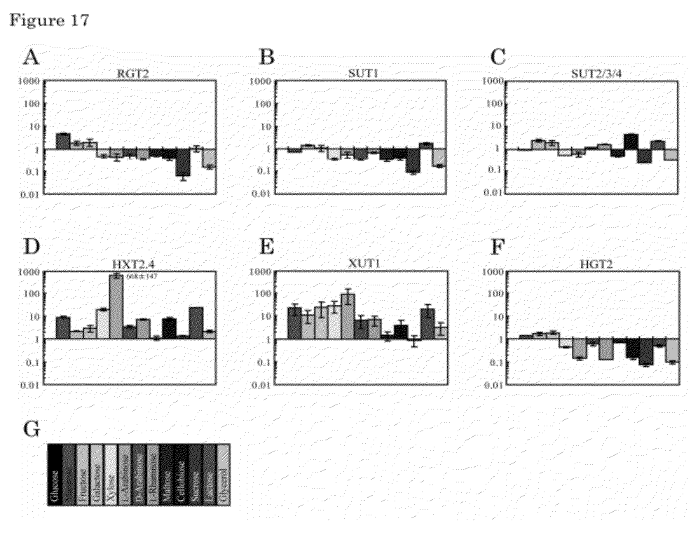

The mean values of two experiments are presented in FIGS. 16 and 17. FIG. 16 shows the expression level of each gene for the different sugars contained in the minimal media. The vertical axis in the graphs represents the expression level of each gene relative to the expression level 1 of the control actin gene used as a house-keeping gene. FIG. 17 summarizes the data presented in FIG. 16 for each expression gene, and represents the expression level of each gene cultured in a minimal medium containing each different sugar at 2% (w/v) concentration, relative to the expression level for glucose contained in the minimal medium at the same concentration.

In any of the hexose minimal media containing glucose, mannose, fructose, and galactose, the highest expression occurred not in the known hexose transporter SUT gene group, but in the HGT2 gene newly identified by the foregoing screening. Further, because expression was sufficient also under growth on the minimal medium containing galactose that cannot be the target of transport by the expression product of the HGT2 gene, it is considered that the HGT2 gene is not induced by a specific sugar, but is constitutively expressed. SUT1 to 4 and RGT2 had the next highest expression after HGT2. This result coincides well with the result of the complementary growth experiment conducted with the KY73 strain.

As to the pentose xylose, it is known from the results of Weierstall et al. (Non-Patent Literature 17) that SUT1 expression is not induced in the presence of xylose, and that SUT2 or SUT3 is expressed only under aerobic conditions. These conventional findings coincide well with the results of this example in which the SUT2 to 4 expression levels were sufficiently lower than the SUT1 expression level in aerobic cultures. On the other hand, HGT2 had expression levels two times or greater than the expression of SUT2 to 4. This, combined with the result of the protein function analysis, suggests that HGT2 is the principal xylose transporter in P. stipitis.

In the experiment using L-arabinose-containing minimal medium, HXT2.4 had the most prominent expression with a level of induction 670 times greater than that in the result of experiment in which a glucose-containing minimal medium was used. However, L-arabinose transportability was not confirmed in the protein (at least in the Saccharomyces yeast), suggesting that the protein may have a problem in the function expression in Saccharomyces yeast. From the foregoing example, sufficient L-arabinose transportability was observed in XUT1 that had the next highest expression level after HXT2.4. This suggests that XUT1 is the principal L-arabinose transporter in P. stipitis. This points to the involvement of HXT2.4 also as an L-arabinose transporter.

### Example 5

### Xylose Transportability in the Presence of Xylose and L-Arabinose

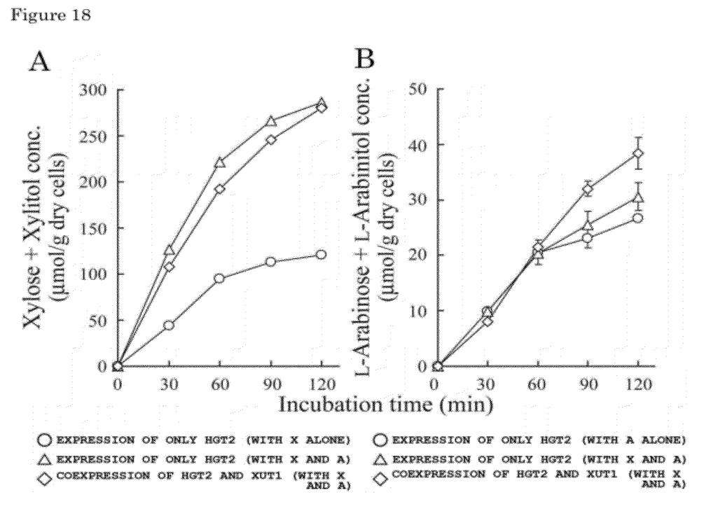

The KY73 strain in which the HGT2 was expressed according to the method of Example 2 or 3 was observed for transportability in the presence of xylose and L-arabinose (each at 2% (w/v) concentration). The xylose uptake improved about three times from that in the absence of L-arabinose (FIG. 18: A). This effect remained unchanged when the HGT2 was expressed with XUT1, and the additive effect of HGT2 and XUT1 was also maintained for the L-arabinose uptake (FIG. 18: B).

In nature, sugars such as xylose and L-arabinose do not exist alone as in laboratories, and typically these sugars are incorporated simultaneously with hexoses produced by, for example, cellulase and hemicellulase. Thus, it is indeed very desirable to have the cooperative effect seen as above in the presence of coexisting sugars such as xylose and L-arabinose in the uptake of xylose and L-arabinose by the yeast cells expressing genes such as HGT2 and XUT1.

### Sequence Listing

PCT_pentose transporter_{—}20100827_{—}214517_{—}0.txt

1. Use of HGT2 gene or an expression product protein thereof as a xylose transporter.

2. (canceled)

3. Use of XUT1 gene as a xylose and/or L-arabinose transporter.

4. A method for producing bioethanol, the method comprising introducing XUT1 gene into yeast, and culturing the yeast in the presence of a biomass that contains xylose and/or L-arabinose.

5. A bioethanol producing method according to claim 4, wherein the biomass containing xylose and/or L-arabinose is a lignocellulose.

6. A method for producing bioethanol, the method comprising introducing HGT2 gene into yeast, and culturing the yeast in the presence of a biomass that contains xylose.

7. A bioethanol producing method according to claim 6, wherein the biomass containing xylose is a lignocellulose.
